(19) United States
(12) Patent Application Publication
(10) Pub. No.: US 2003/0139792 A1
(43) Pub. Date: Jul. 24, 2003

(54) Fistula thermotherapy device

(76) Inventors: Kuo-Hsin Ho, Taipei Hsien (TW); Liang-Kuang Diang, Taipei Hsien (TW)

Correspondence Address: BIRCH STEWART KOLASCH & BIRCH, PO BOX 747, FALLS CHURCH, VA, 22040-0747, US

(21) Appl. No.: 10/053,579

(22) Filed: Jan. 24, 2002

(51) Int. Cl. A61F 7/00

(52) U.S. Cl. 607/108

(57) ABSTRACT

A fistula thermotherapy device uses 4-14 μm infrared waves for thermotherapy on fistula. The infrared waves penetrate the epidermis of patients 2-3 centimeters deep so as to have sanative effect on fistula and cells nearby. Therefore, the blood circulation of fistula is improved, the cells nearby are activated, and the metabolism of that area is enhanced. Thus the fluency and elasticity of the fistula can be maintained. The fistula thermotherapy device includes an electric heating layer, an inner cover, and an outer cover.

### BACKGROUND OF THE INVENTION

1. Field of the Invention

The present invention relates to a fistula thermotherapy device, especially to a fistula thermotherapy device that not only protects the arm with the fistula for hemodialysis, but also facilitate the metabolism of the cells near the needle insertions on fistula. Moreover, the fluency and the elasticity of the fistula are maintained.

2. Description of the Prior Art

Before starting hemodialysis treatments, one important step is preparing a vascular access, which is the site on your body where blood will be removed and returned during dialysis. For most people, an arteriovenous (AV) fistula is the best kind of vascular access. An AV fistula is created by connecting an artery directly to a vein, usually in the forearm of patients. Then the vein grows larger and stronger, making repeated needle insertions easier. The blood tube for needle insertions is called fistula.

However, the regular hemodialysis sessions take 2-3 times per week. In the long run, there is hundreds of pinholes concentrate on the fistula that is only several centimeters long. Thus the fistula is likely to form clots or get infected caused by repeatedly usage. Finally the fistula needs replacement sooner. The general way for protecting the arm with fistula thereof is to avoid heavy loading, the fistula can only be used for hemodialysis, no pressing on the area, or taking more exercise for increasing blood circulation. Such kind of methods can only protect the surface of the area without improving the fistula itself. Once the patients can't follow the above instruction, the fistula for needle insertion is easy to become scabbed or have obstruction. Sooner the complications cause the replacement of fistula. The patients need to prepare a new vascular access again.

### SUMMARY OF THE INVENTION

It is therefore a primary object of the present invention to provide a fistula thermotherapy device that produces infrared waves penetrating the epidermis of patients 2-3 centimeters deep so as to have sanative effect on fistula and cells nearby. Therefore, the blood circulation of fistula is improved, the cells nearby are activated, the metabolism of that area is enhanced.

It is a further object of the present invention to provide a fistula thermotherapy device whose outer cover is made of a layer of absorptive shag so as to keep moisture on the skin nearby the fistula while having thermotherapy. Thus the fistula tends to last longer.

### BRIEF DESCRIPTION OF THE DRAWINGS

The accomplishment of the above-mentioned object of the present invention will become apparent from the following description and its accompanying drawings which disclose illustrative an embodiment of the present invention, and are as follows:

FIG. 1 is an explosive view of the present invention;

FIG. 2 is a sectional view of a heating wire in accordance with the present invention;

FIG. 3 is an embodiment of the present invention.

### DETAILED DESCRIPTION OF THE PREFERRED EMBODIMENT

The present invention—fistula thermotherapy device—uses 4-14 μm infrared waves for thermotherapy in order to increase the blood circulation, activate cells, facilitate the metabolism of the fistula. Thus the fluency and elasticity of fistula can be maintained.

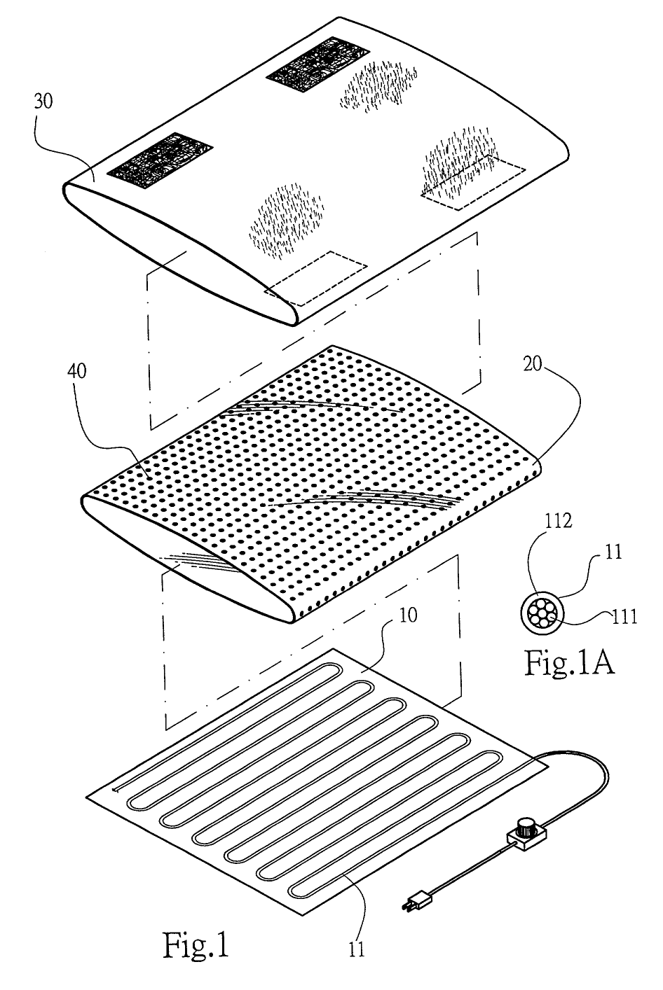

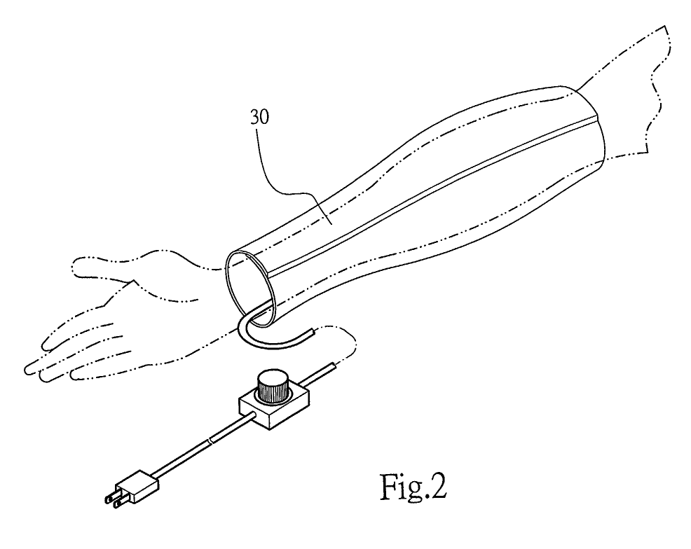

Refer to FIG. 1 & FIG. 2, the fistula thermotherapy device is composed of an electric heating layer 10, an inner cover 20, and an outer cover 30. The electric heating layer 10 includes heating wire 11 made of graphite heating element 111 (as shown in FIG. 1A) with a covering layer 112 surrounding. The inner cover 20 is made from heatproof and tension-proof artificial fiber coated with colloid granules 40 which are covered with infrared waves powder. The outer cover 30 is made of shag that draws moisture from humidity in the air.

In accordance with the structure mentioned above, the present invention covers on the arm of hemodialysis patients that has fistula. When current flows in, the heating layer 10 heats the colloid granule 40 on the surface of the inner cover 20 so as to produce infrared waves. The wavelength of 70% of the infrared waves produced is between 4-14 μm and the infrared waves penetrate the epidermis of patients 2-3 centimeters deep so as to have sanative effect on fistula and cells nearby. Therefore, the blood circulation of fistula is improved, the cells nearby are activated, the metabolism of that area is enhanced.

The colloid granule 40 with infrared waves powder on the surface of the inner cover 20 can also be a covering layer 112 on the heating wire 11 of the electric heating layer 10 or covers on the surface of the covering layer 112.

As to the outer cover 30 made of the absorptive shag draws moisture released from human bodies or in the air. When patients put the present invention on the arm with fistula for thermotherapy, the heat energy from infrared waves evaporate the water inside the outer cover 30 so as to protect the skin nearby the fistula from drying while having thermotherapy.

The present invention not only has simple structure, convenient for use but also achieves the sanative effect on fistula under epidermis.

It should be noted that the above description and accompanying drawings are only used to illustrated some embodiments of the present invention, not intended to limit the scope thereof. Any modification of the embodiments should fall within the scope of the present invention.

What is claimed is:

1. A fistula protective device comprising an electric heating layer, an inner cover, and an outer cover, wherein said electric heating layer includes heating wire having graphite heating element and a covering layer surrounding; said inner cover is made from heatproof and tension-proof artificial fiber coated with colloid granules which are covered with infrared waves powder thereof; said outer cover is made of absorptive shag; when being used, the fistula protective device covers on the arm of patients, heating layer heats said colloid granule on the surface of said inner cover so as to produce infrared waves that penetrate the epidermis of patients 2-3 centimeters deep for thermotherapy.

2. A fistula protective device as claimed in claim 1, wherein said colloid granule with infrared waves powder on the surface of the inner cover can also be used as said covering layer on said heating wire of said electric heating layer or covers directly on the surface of said covering layer.
